(19) United States
(12) Patent Application Publication
(10) Pub. No.: US 2025/0147005 A1
(43) Pub. Date: May 8, 2025

(54) INTESTINAL INFORMATION ESTIMATION SYSTEM

(71) Applicant: KYOCERA Corporation, Kyoto-shi, Kyoto (JP)

(72) Inventors: Shingo TERANISHI, Kyoto-shi, Kyoto (JP); Daisuke UEYAMA, Kyoto-shi, Kyoto (JP); Shinichi ABE, Kyoto-shi, Kyoto (JP)

(21) Appl. No.: 18/832,868

(22) PCT Filed: Jan. 20, 2023
(86) PCT No.: PCT/JP2023/001617

§ 371 Date: Jul. 24, 2024

(30) Foreign Application Priority Data
Jan. 27, 2022 (JP) 2022-011294

(51) Int. Cl. G01N 33/497

(52) U.S. Cl. CPC G01N 33/4975

(57) ABSTRACT

Intestinal information of a subject is accurately estimated from a component contained in a gas generated from feces of the subject. Included are a detector configured to detect a predetermined component from a gas released from feces of a subject and output a detection signal depending on a concentration of the predetermined component and an estimator configured to input the detection signal or the concentration of the predetermined component corresponding to the detection signal to a prediction model and estimate information regarding an amount and/or an existence ratio of short-chain fatty acid-producing bacteria and/or a metabolite contained in the feces of the subject. The predetermined component is at least one selected from the group consisting of methyl mercaptan, hydrogen sulfide, hydrogen, and carbon dioxide.

### TECHNICAL FIELD

The present disclosure relates to an intestinal information estimation system for estimating intestinal information of a subject.

### BACKGROUND OF INVENTION

Patent Document 1 describes an intestinal condition notification device which notifies a user of information regarding balance of enterobacteria corresponding to a signal value output from a gas sensor that detects a predetermined gas component within excreted gas. The intestinal condition notification device stores corresponding data which show a correspondence relation between the signal value output from the gas sensor and information regarding balance of enterobacteria of a user and notifies the user of information regarding balance of enterobacteria corresponding to the signal value output from the gas sensor based on the corresponding data.

### CITATION LIST

### Patent Literature

Patent Document 1: JP 2007-89857 A

### SUMMARY

An intestinal information estimation system according to an aspect of the present disclosure includes: a detector configured to detect a predetermined component from a gas released from feces of a subject and output a detection signal depending on a concentration of the predetermined component; and an estimator configured to input the detection signal or the concentration of the predetermined component corresponding to the detection signal to a prediction model and estimate information regarding an amount and/or an existence ratio of short-chain fatty acid-producing bacteria and/or a metabolite contained in the feces of the subject. The predetermined component is at least one selected from the group consisting of methyl mercaptan, hydrogen sulfide, hydrogen, and carbon dioxide.

### BRIEF DESCRIPTION OF THE DRAWINGS

FIG. 1 is a schematic diagram illustrating an example of a configuration of an intestinal information estimation system according to an embodiment.

FIG. 2 is a chart illustrating an example of a data structure of detection information.

FIG. 3 is a chart illustrating an example of a data structure of detection data.

FIG. 4 is a chart illustrating an example of a data structure of subject information.

FIG. 5 is a chart illustrating an example of a data structure of estimation result information.

FIG. 6 is a chart illustrating an example of a data structure of intestinal information.

FIG. 7 is a chart illustrating an example of a data structure of health information.

FIG. 8 is a diagram illustrating an appearance of a gas detection device included in the intestinal information estimation system illustrated in FIG. 1.

FIG. 9 is a block diagram illustrating a configuration of a main part of the intestinal information estimation system.

FIG. 10 is a schematic diagram illustrating an example of a configuration of intestinal information estimation.

FIG. 11 is a flowchart showing an example of a flow of processing performed in the intestinal information estimation system.

FIG. 12 is a chart in which a butyric acid amount estimated from a concentration of H₂S is plotted.

FIG. 13 is a chart in which a Ruminococcus bacteria ratio estimated from a concentration ratio of CH₃SH is plotted.

FIG. 14 is a chart in which a glucose 6-phosphate amount estimated from a ratio of H₂S and CH₃SH is plotted.

FIG. 15 is a chart in which a ratio of a sum of Faecalibacteria and Lachnospira bacteria estimated from a ratio of H₂S to CH₃SH is plotted.

FIG. 16 is a chart in which a Faecalibacteria ratio estimated from a CH₃SH concentration is plotted.

FIG. 17 is a chart in which a Ruminococcus bacteria ratio estimated from a CH₃SH concentration is plotted.

FIG. 18 is a chart in which a Lachnospira bacteria ratio estimated from a ratio of CH₃SH is plotted.

FIG. 19 is a chart in which an ornithine amount estimated from a CH₃SH concentration is plotted.

FIG. 20 is a chart in which a trimethylamine amount estimated from a ratio of CH₃SH is plotted.

FIG. 21 is a chart in which a Streptococcus bacteria ratio estimated from a ratio of CH₃SH is plotted.

FIG. 22 is a chart in which a ratio of bifidobacteria estimated from a CO₂ concentration is plotted.

FIG. 23 is a chart in which an ornithine amount estimated from a CH₃SH concentration is plotted.

FIG. 24 is a chart in which a Coprococcus bacteria ratio estimated from a CO₂ concentration is plotted.

FIG. 25 is a diagram illustrating an example of an appearance of an intestinal information estimation device 1.

FIG. 26 is a schematic diagram illustrating a variation of the intestinal information estimation system.

### DESCRIPTION OF EMBODIMENTS

It is necessary to accurately estimate intestinal information of a subject from a component contained in a gas generated from feces of the subject.

An aspect of the present disclosure can accurately estimate intestinal information of a subject from a component contained in a gas generated from feces of the subject.

### First Embodiment

The inventors have found that information regarding an inside of an intestine of a subject can be obtained by analyzing concentrations of methyl mercaptan, hydrogen sulfide, hydrogen, and carbon dioxide detected from a sample gas released from feces of the subject.

The inventors have developed an intestinal information estimation system 100 configured to estimate information regarding an amount and/or an existence ratio of short-chain fatty acid-producing bacteria and/or a metabolite contained in feces from a concentration of a predetermined component detected from a gas released from feces of a subject.

The “subject” is a person who uses the intestinal information estimation system 100 described later and intended to be a subject whose health condition is managed and monitored. The “sample gas” is a gas of a detection target and is defecation gas of the subject.

The intestinal information estimation system 100 according to an aspect of the present disclosure detects a predetermined component from a gas released from feces of a subject, outputs a detection signal depending on a concentration of the predetermined component, and inputs the detection signal or the concentration of the predetermined component corresponding to the detection signal to a prediction model. Thus, the intestinal information estimation system 100 is a system that can estimate information regarding an amount and/or an existence ratio of short-chain fatty acid-producing bacteria and/or a metabolite contained in feces of the subject.

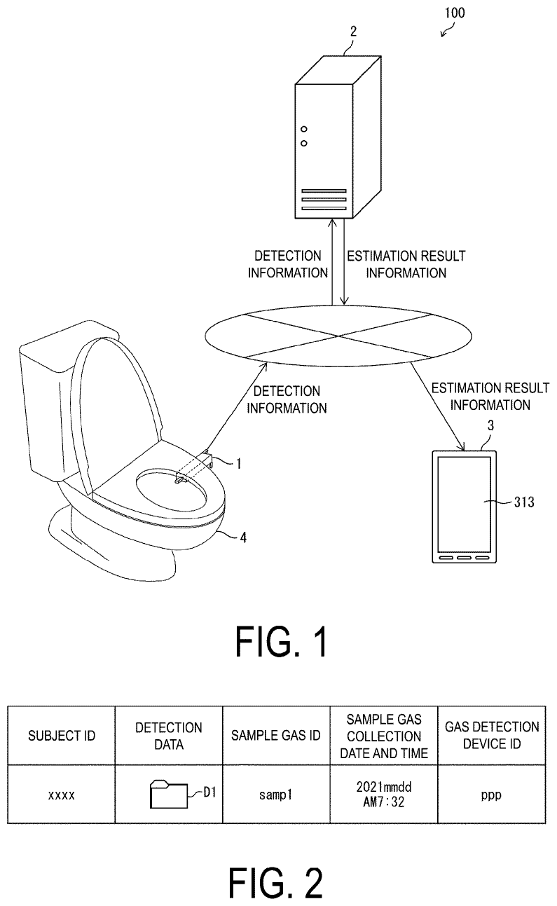

As illustrated in FIG. 1, the intestinal information estimation system 100 may be a system configured to detect a predetermined component from a gas released from feces of the subject in a toilet, as an example. In this case, a gas detection device 1 for detecting a predetermined component from a gas, described later, may be installed in a toilet 4 of a bathroom. According to the above configuration, the intestinal information estimation system 100 performs processing of detecting a predetermined component from a gas released from feces of a subject in a bathroom. Therefore, the user who uses the intestinal information estimation system 100 does not need to perform troublesome work such as feces examination and simply uses the bathroom.

As illustrated in FIG. 25, the intestinal information estimation system 100 may be a system configured to detect a predetermined component from a gas released from feces of a subject on a bed 5 for a person requiring nursing care, as an example. In this case, the gas detection device 1 (detector 102) for detecting a predetermined component from a gas, described later, may be installed in the bed 5 for a person requiring nursing care. As illustrated in FIG. 25, when the bed 5 and a toilet 4C are integrated, the gas detection device 1 may be installed in the toilet 4C. According to the above configuration, the intestinal information estimation system performs processing of detecting a predetermined component from a gas released from feces of a subject on the bed for a person requiring nursing care. This can make the subject who is a person requiring nursing care use the intestinal information estimation system 100 without difficulty.

The detector 102 of the intestinal information estimation system 100 needs not be fixedly installed at one place and may be carried by the subject, for example. Specifically, the intestinal information estimation system may have an aspect in which the subject carries the gas detection device 1 of the intestinal information estimation system 100 and attaches the gas detection device 1 to the toilet of the bathroom for use every time the subject uses the bathroom. According to the above configuration, the user can use the intestinal information estimation system 100 at any place (e.g., a place where the user goes out or the like).

### <Configuration of Intestinal Information Estimation System 100>

An embodiment of the present disclosure will be described in detail below. Hereinafter, as an example, a system in which the intestinal information estimation system 100 detects a predetermined component in a bathroom will be described. FIG. 1 is a schematic diagram illustrating an example of the configuration of the intestinal information estimation system 100 according to an embodiment of the present disclosure. Each of the figures referred to in the present description is a schematic diagram illustrating only some members in a simplified manner in order to describe the embodiment for convenience of description. Therefore, the intestinal information estimation system 100 can include any constituent member not illustrated in the figures to which present description refers. The dimensions of members in the respective figures do not accurately represent the actual dimensions of constituent members, the dimensional ratio of respective members, or the like.

The intestinal information estimation system 100 includes the gas detection device 1, an intestinal information estimation device 2, and an electronic device 3. In the intestinal information estimation system 100, the gas detection device 1, the intestinal information estimation device 2, and the electronic device 3 may be communicably connected to one another. The gas detection device 1 and the intestinal information estimation device 2, and the electronic device 3 and the intestinal information estimation device 2 may be connected by wireless communication or wired communication.

### Gas Detection Device 1

The gas detection device 1 detects a predetermined component from a gas released from feces of a subject and outputs a detection signal depending on the concentration of the predetermined component. The gas detection device 1 may calculate the concentration of the predetermined component corresponding to the detection signal and output information on the calculated concentration. Here, the information output by the gas detection device 1 is called “detection information”. The gas detection device 1 transmits the detection information to the intestinal information estimation device 2.

### Detection Information

Detection information output from the gas detection device 1 will be described with reference to FIG. 2. FIG. 2 is a chart illustrating an example of the data structure of detection information output from the gas detection device 1. As illustrated in FIG. 2, the detection information may include a subject ID, detection data D1, a sample gas ID, and a sample gas collection date and time.

The subject ID is identification information unique to the subject. The subject ID may be a name of the subject and identification information unique to each subject. When the subject is a user who uses the intestinal information estimation system 100, the subject ID may be a user ID assigned to each user who uses the intestinal information estimation system 100.

The gas detection device 1 may collect a sample gas a plurality of times at predetermined time intervals (e.g., 30 seconds, 1 minute, or the like) for one defecation of the subject. A sample gas ID may be assigned to each collected sample gas. FIG. 2 exemplifies detection information output from the gas detection device 1 used by the subject with a subject ID “xxxx”. As an example, a sample ID “samp1” is assigned to a sample gas collected at “7:32 AM, on the month of mm, the day of dd, 2021”.

The detection data D1 may include data indicating the concentration of a predetermined component for each sample based on a detection signal output by the detector 102. The predetermined component contains at least one selected from the group consisting of methyl mercaptan (CH₃SH), hydrogen sulfide (H₂S), hydrogen (H₂), and carbon dioxide (CO₂). The predetermined component may further contain 2-propanol. The detection data D1 may be a detection signal output from the detector 102 or a numerical value indicating the concentration calculated from the detection signal. Here, the concentration of a predetermined component may be the concentration of the predetermined component in a gas collected by the gas detection device 1. The predetermined component may contain a plurality of components, and the concentration may be a concentration of a sum of the plurality of components with respect to the total amount of the sample gas. The unit of concentration may be ppm, for example.

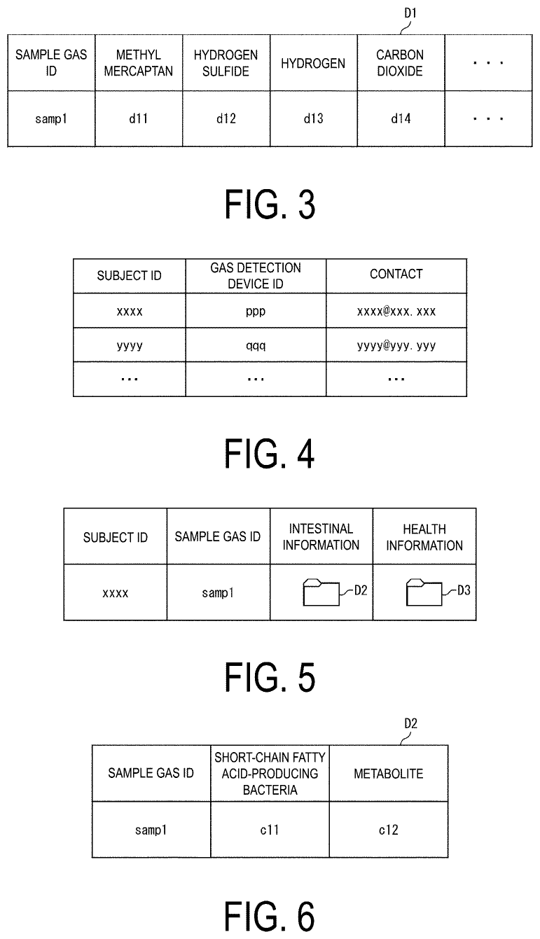

FIG. 3 is a chart illustrating an example of the data structure of the detection data D1. As illustrated in FIG. 3, the detection data D1 may include the following detected from the sample gas of the sample ID “samp1”.
Concentration d11 of methyl mercaptan
Concentration d12 of hydrogen sulfide
Concentration d13 of hydrogen
Concentration d14 of carbon dioxide
   The detection information may further include a gas detection device ID unique to the gas detection device 1. FIG. 2 illustrates detection information including a gas detection device ID “ppp” of the gas detection device 1 used by the subject with the subject ID “xxxx”, as an example.

### Intestinal Information Estimation Device 2

The intestinal information estimation device 2 illustrated in FIG. 1 may be a computer managed by a manager of the intestinal information estimation system 100 or may be a server device. The intestinal information estimation device 2 inputs a detection signal acquired from the gas detection device 1 or the concentration of a predetermined component corresponding to the detection signal to a prediction model. The intestinal information estimation device 2 estimates information regarding an amount and/or an existence ratio of short-chain fatty acid-producing bacteria and/or a metabolite contained in feces of the subject. That is, the intestinal information estimation device 2 estimates intestinal information regarding the intestinal environment of the subject. Information output by the intestinal information estimation device 2 is called “estimation result information”.

The short-chain fatty acid-producing bacteria estimated by the intestinal information estimation device 2 are a kind of intestinal bacteria and are bacteria that produce short-chain fatty acid. Specifically, the short-chain fatty acid-producing bacteria may be butyric acid-producing bacteria and/or acetic acid-producing bacteria.

Examples of the butyric acid-producing bacteria include Faecalibacteria, Lachnospira bacteria, and Coprococcus bacteria.

Examples of the acetic acid-producing bacteria include bifidobacteria.

The metabolite estimated by the intestinal information estimation device 2 may be a substance involved in the metabolic system of intestinal bacteria of the subject. Examples of the metabolite include butyric acid, acetic acid, ornithine, trimethylamine, and glucose 6-phosphate.

For example, the intestinal information estimation device 2 may hold subject information in which the ID of each subject, the gas detection device ID of the gas detection device 1 used by each subject, and the contact of each subject are associated.

FIG. 4 is a chart illustrating an example of the data structure of subject information held in the intestinal information estimation device 2. The contact of the subject may be an email address of the subject. With reference to the subject information, the intestinal information estimation device 2 specifies the subject who uses the gas detection device 1 that is a transmission source of the detection information from the subject ID included in the detection information and transmits the estimation result information to the electronic device 3 of the subject. The subject information illustrated in FIG. 4 indicates that the gas detection device ID of the gas detection device 1 used by the subject with the subject ID “xxxx” is “ppp”, and the contact of the subject is “xxxx/@xxx.xxx”.

Alternatively, the intestinal information estimation device 2 may be configured to create a web page unique to each subject and allow each subject to browse this web page. Each subject may be caused to set a unique password or the like for browsing an own web page. In this case, with reference to the subject information, the intestinal information estimation device 2 specifies the subject from the subject ID and transmits the URL or the like of the web page to the electronic device 3 of the subject.

The intestinal information estimation device 2 may include a function of estimating the health condition of the subject from the intestinal information.

### Estimation Result Information

The estimation result information will be described with reference to FIG. 5. FIG. 5 is a chart illustrating an example of the data structure of the estimation result information. As illustrated in FIG. 5, the estimation result information may include a subject ID, a sample gas ID, intestinal information D2, and health information D3.

FIG. 6 is a chart illustrating an example of the data structure of the intestinal information D2. As illustrated in FIG. 6, the intestinal information D2 includes information regarding an amount or existence ratio c11 of short-chain fatty acid-producing bacteria and an amount or existence ratio c12 of a metabolite.

Here, the amount of the short-chain fatty acid-producing bacteria may be the number of short-chain fatty acid-producing bacteria contained in feces of the subject of a predetermined mass or may be the mass of the short-chain fatty acid-producing bacteria. The unit of the amount may be, for example, “piece”, “g”, or “mg”.

The existence ratio of the short-chain fatty acid-producing bacteria may be a ratio to the total number of the short-chain fatty acid-producing bacteria contained in feces of the subject of a predetermined mass. The existence ratio of the short-chain fatty acid-producing bacteria may be, for example, the sum of the masses of two or more short-chain fatty acid-producing bacteria contained in the feces of the subject of a predetermined mass.

The amount of the metabolite may be the mass or molecular weight of the metabolite contained in feces of the subject of a predetermined mass. The existence ratio of the metabolite may be a ratio to the total mass of the metabolite contained in feces of the subject of a predetermined mass. The existence ratio of the metabolite may be, for example, the sum of the masses of two or more metabolites contained in feces of the subject of a predetermined mass. The unit of amount may be, for example, “g” or “mg”.

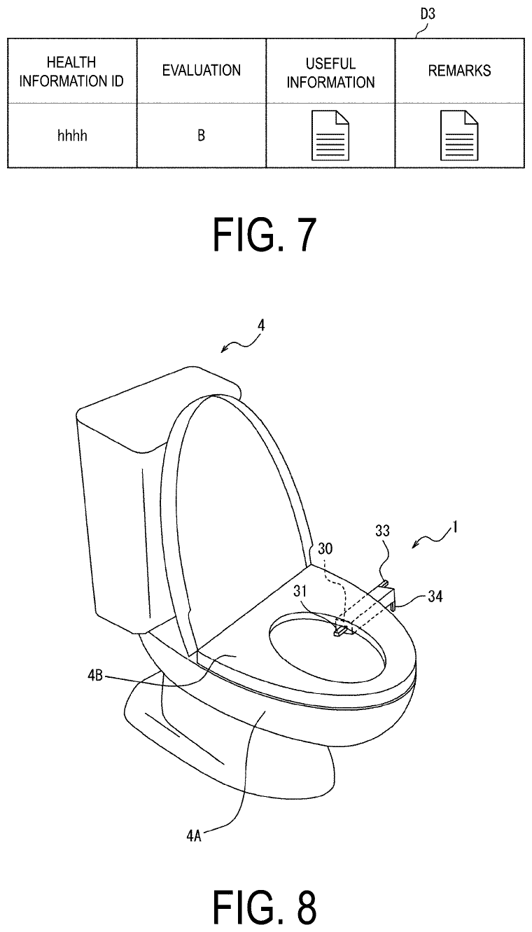

FIG. 7 is a chart illustrating an example of the data structure of the health information D3. As illustrated in FIG. 7, the health information D3 may include evaluation, useful information, and remarks. The health information D3 may include a health information ID assigned to each piece of health information.

The evaluation may be a determination result of the health condition of the subject estimated by the intestinal information estimation device 2 based on the amount or existence ratio c11 of the short-chain fatty acid-producing bacteria and the amount or existence ratio c12 of the metabolite. The evaluation may be a determination result regarding the state of an intestinal bacterial flora (also called an intestinal flora) of the subject estimated based on the amount or existence ratio c11 of the short-chain fatty acid-producing bacteria and the amount or existence ratio c12 of the metabolite. For example, a three-stage determination of A (good), B (allowable range), and C (caution needed) may be applied to the evaluation of the health condition of the subject. FIG. 7 illustrates an example in which the health condition of the subject is evaluated as “B”.

The useful information may be useful information that contributes to improvement of the health condition of the subject. The useful information may include information regarding foods (ingredients and dishes) and exercise recommended to the subject and information regarding lifestyle improvement.

The remarks may include various information provided to the subject. The remarks may include, for example, the following information.
Contact of a dietitian whom the subject can consult on health issues.
Access information to a video introducing a cooking method of a dish using a recommended ingredient.
Information on online shopping websites where ingredients and exercise equipment can be purchased.

### Electronic Device 3

Returning to FIG. 1, the electronic device 3 may be a computer used by the subject. Alternatively, the electronic device 3 may be a computer used by a person (e.g., a family member or the like) who monitors the health condition of the subject. The electronic device 3 may be, for example, a personal computer, a tablet terminal, a smartphone, or the like.

The electronic device 3 has a communication function and can receive the estimation result information from the intestinal information estimation device 2. The electronic device 3 may include, for example, an input unit such as a keyboard, a touchscreen, and a microphone, and a display such as a monitor. The electronic device 3 may be installed inside a bathroom where the toilet 4 is installed. In this case, the electronic device 3 may be able to be taken out to the outside of the bathroom.

### Gas Detection Device 1

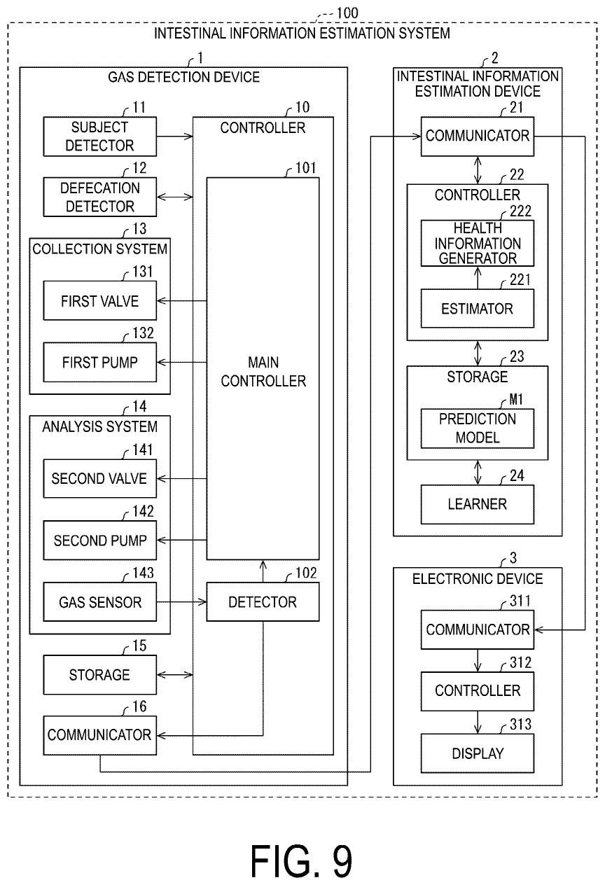

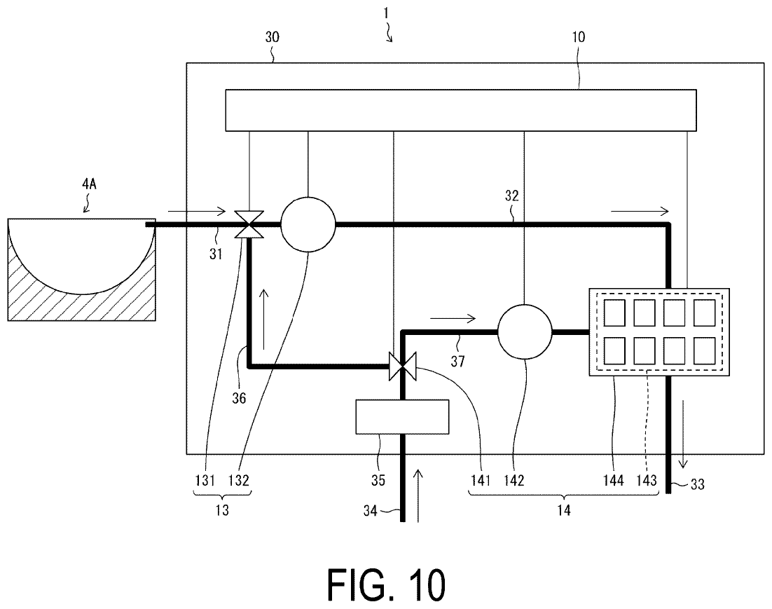

As described above, the gas detection device 1 is a device configured to collect a sample gas released from feces of a subject, detect a predetermined component from each collected sample gas, and output a detection signal depending on the concentration of the predetermined component. The gas detection device 1 may collect a sample gas and detect a predetermined component a plurality of times and may transmit a detection result to the intestinal information estimation device 2 based on each result. Hereinafter, the gas detection device 1 will be described with reference to FIGS. 8 to 10. FIG. 8 is a diagram illustrating an appearance of the gas detection device 1 included in the intestinal information estimation system 100. FIG. 9 is a block diagram illustrating the configuration of a main part of the intestinal information estimation system 100 illustrated in FIG. 1. FIG. 10 is a schematic diagram illustrating an example of the configuration of the gas detection device 1.

As illustrated in FIG. 8, the gas detection device 1 is installed in the flush toilet 4, for example. The toilet 4 includes a toilet bowl 4A and a toilet seat 4B. The toilet 4 can be installed in a bathroom of a house, a hospital, or the like. The gas detection device 1 may be installed at any position of the toilet 4. As an example, as illustrated in FIG. 8, the gas detection device 1 may be disposed from between the toilet bowl 4A and the toilet seat 4B to the outside of the toilet 4. A part of the gas detection device 1 may be embedded in the toilet seat 4B. Feces of the subject can be excreted to the toilet bowl 4A of the toilet 4. The gas detection device 1 can acquire a sample gas in which a gas generated from feces excreted to the toilet bowl 4A is mixed with outside air. The gas detection device 1 can detect the type, concentration, and the like of a predetermined component contained in the sample gas.

As illustrated in FIG. 9, the gas detection device 1 includes a controller 10, a subject detector 11, a defecation detector 12, a collection system 13, an analysis system 14, a storage 15, and a communicator 16. The controller 10 controls the operation of each part of the gas detection device 1 and detects each detected gas contained in the sample gas. Details of the controller 10 will be described later.

The subject detector 11 may include at least any of an image camera, an individual identification switch, an infrared sensor, and a pressure sensor. The subject detector 11 outputs the detection result to the controller 10. The subject detector 11 may include any sensor for authenticating the subject. Examples of the sensors include a load sensor configured to detect body weight, a sensor configured to detect seating height, a sensor configured to detect a pulse, a sensor configured to detect a blood flow, a sensor configured to detect a face, and a sensor configured to detect voice.

For example, in a case of including an infrared sensor, the subject detector 11 can detect that the subject has entered the bathroom by detecting reflected light from the object of the infrared light emitted by the infrared sensor. As a detection result, the subject detector 11 outputs, to the controller 10, a signal indicating that the subject has entered the bathroom.

For example, in a case of including a pressure sensor, the subject detector 11 can detect that the subject sits on the toilet seat 4B by detecting the pressure applied to the toilet seat 4B as illustrated in FIG. 8. As a detection result, the subject detector 11 outputs, to the controller 10, a signal indicating that the subject sits on the toilet seat 4B.

For example, in a case of including a pressure sensor, the subject detector 11 can detect that the subject has risen from the toilet seat 4B by detecting a decrease in the pressure applied to the toilet seat 4B as illustrated in FIG. 8. As a detection result, the subject detector 11 outputs, to the controller 10, a signal indicating that the subject has risen from the toilet seat 4B.

For example, in a case of including an image camera and an individual identification switch, the subject detector 11 gathers data such as a face image, sitting height, and body weight. The subject detector 11 specifies and detects an individual from the gathered data. As a detection result, the subject detector 11 outputs, to the controller 10, a signal indicating the specified and identified individual.

For example, in a case of including an individual identification switch, the subject detector 11 specifies (detects) an individual based on the operation of the individual identification switch. In this case, personal information may be registered (stored) in advance in the controller 10. As a detection result, the subject detector 11 outputs, to the controller 10, a signal indicating the specified individual.

The defecation detector 12 is a member configured to detect excretion (defecation) of a specimen (feces) from the subject. The defecation detector 12 starts operation in accordance with the control of a main controller 101, and upon detecting that the specimen has been excreted to the toilet bowl 4A, outputs, to the controller 10, a signal indicating that the specimen has been excreted to the toilet bowl 4A. For example, the defecation detector 12 may be a sensor configured to detect a sound when the specimen slips under the water stored in the toilet bowl 4A. In this case, the defecation detector 12 outputs, to the controller 10, a signal indicating information indicating the detected sound. Alternatively, the defecation detector 12 may be a pressure sensor that can detect that the specimen has fallen into the toilet bowl 4A.

The collection system 13 sucks (collects) and stores the sample gas together with outside air from a space in the toilet bowl 4A. Details of the collection system 13 will be described later. The analysis system 14 detects the type and concentration of each detected gas contained in the sample gas using the sample gas collected by the collection system 13. Details of the analysis system 14 will be described later.

The storage 15 includes, for example, a semiconductor memory or a magnetic memory. The storage 15 stores various kinds of information, a program for operating the gas detection device 1, and the like. The storage 15 may function as a work memory. The storage 15 may store an estimation model used for various estimations performed in the controller 10.

The communicator 16 may be communicable with the intestinal information estimation device 2. The communication method used in the communication between the communicator 16 and the intestinal information estimation device 2 may be a short-range wireless communication standard, a wireless communication standard for connection to a mobile phone network, or a wired communication standard. The short-range wireless communication standard may include, for example, Wi-Fi (registered trademark), Bluetooth (registered trademark), infrared rays, and near field communication (NFC). The wireless communication standard for connection to the mobile phone network may include, for example, long term evolution (LTE) or a mobile communication system of a fourth generation or higher. The communication method used in the communication between the communicator 16 and the intestinal information estimation device 2 may be a communication standard such as low power wide area (LPWA) or low power wide area network (LPWAN), for example.

### Collection System 13

Details of the collection system 13 will be described below. As illustrated in FIG. 10, the collection system 13 includes a first valve 131 and a first pump 132. As illustrated in FIG. 10, each part of the collection system 13 is connected by a flow path 31 and a flow path 32.

The first valve 131 included in the collection system 13 is a valve that is located on the flow path 31 and operates in accordance with the control of the main controller 101. The first valve 131 may be configured by a valve that is electromagnetically driven, piezo driven, motor driven, or the like. By adjusting the degree of opening (degree of communication) of each flow path in accordance with the control of the main controller 101, the first valve 131 can adjust communication states between the flow path 31 and the flow path 32 and between the flow path 32 and a flow path 36 (described later). Hence, inflow of the sample gas and a purge gas into the flow path and a sensor chamber 144 (described later) can be adjusted.

The first pump 132 is provided between the flow path 31 and the flow path 32 and is connected to the sensor chamber 144 via the flow path 32. The first pump 132 operates under the control of the main controller 101. The first pump 132 sucks the sample gas in the toilet bowl 4A through an opening of the flow path 31 opened toward the inside of the toilet bowl 4A and supplies the sample gas to the flow path 32. The first pump 132 illustrated in FIG. 10 may include a piezo pump or a motor pump. As described later, the first pump 132 may also be used when the flow path 32 is supplied with the purge gas.

The flow path 31 is a tubular member provided for connecting the toilet bowl 4A and the first pump 132. One end of the flow path 31 has an opening that opens in the toilet bowl 4A, and the opposite end is connected to the first pump 132. The flow path 32 is a flow path provided between the first pump 132 and the sensor chamber 144. When the first pump 132 operates in a state where the first valve 131 is opened, a gas can be supplied from the flow path 31 or the flow path 36 (described later) to the flow path 32.

### Analysis System 14

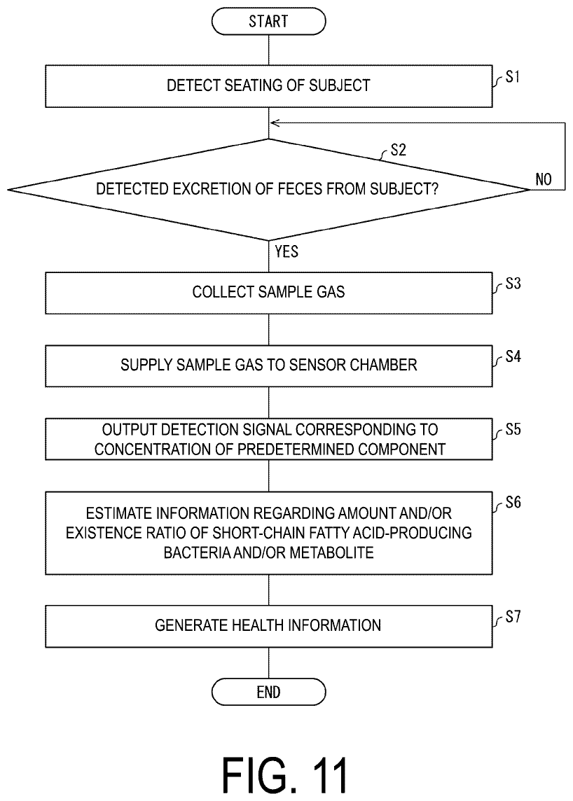

Details of the analysis system 14 will be described below. As illustrated in FIG. 10, the analysis system 14 includes a second valve 141, a second pump 142, a gas sensor 143, and the sensor chamber 144. As illustrated in FIG. 11, the analysis system 14 is connected to the outside by a discharge path 33 and a flow path 34. Parts of the analysis system are connected by a flow path 37.

The second valve 141 is a valve provided on the flow path 34. The second valve 141 operates in accordance with the control of the main controller 101 and can switch between a state in which the flow path 34 and the flow path 36 communicate with each other and a state in which the flow path 34 and the flow path 37 communicate with each other.

The second pump 142 is a pump provided on the flow path 37 and connected to the sensor chamber 144 via the flow path 37. The second pump 142 operates under the control of the main controller 101 and can supply the sensor chamber 144 with the outside air sucked from the flow path 34.

The gas sensor 143 may be a sensor configured to output different detection signals depending on the concentration of the detected gas. Hereinafter, as the gas sensor 143, a sensor in which the intensity of the detection signal changes in accordance with the concentration of the detected gas will be described as an example, but the gas sensor is not limited to this. As an example, the gas sensor 143 can output a detection signal having an intensity corresponding to the concentration of the detected gas that can be contained in the sample gas. As illustrated in FIG. 10, a plurality of gas sensors 143 may be located in the gas detection device 1. The plurality of gas sensors 143 may be able to output detection signals corresponding to the concentrations of different types of detected gases. This enables the gas detection device 1 to analyze the concentrations of a plurality of types of detected gases.

The gas sensor 143 includes a sensor element and a resistance element. The sensor element and the resistance element are connected in series between a power supply terminal and a ground terminal. A constant voltage value VC is applied between the power supply terminal and the ground terminal. A same current value IS flows through each of the sensor element and the resistance element. The current value IS can be determined in accordance with a resistance value RS of the sensor element and a resistance value RL of the resistance element. The voltage output from the gas sensor 143 may be a voltage value VS applied to the sensor element or a voltage value VRL applied to the resistance element.

The power supply terminal is connected to a power supply such as a battery included in the gas detection device 1. The ground terminal is connected to the ground of the gas detection device 1. One end of the sensor element is connected to the power supply terminal. The opposite end of the sensor element is connected to one end of the resistance element. As an example, the sensor element is a semiconductor sensor. However, the sensor element is not limited to the semiconductor sensor. For example, the sensor element may be a contact combustion sensor, a solid electrolyte sensor, or the like.

The sensor element includes a gas sensing portion. The gas sensing portion includes a metal oxide semiconductor material corresponding to the type of the gas sensor 143. Examples of the metal oxide semiconductor material include a material containing one or more types selected from tin oxide (SnO₂ or the like), indium oxide (In₂O₃ or the like), zinc oxide (ZnO or the like), tungsten oxide (WO; or the like), iron oxide (Fe₂O₃ or the like), and the like. By appropriately adding impurities to the metal oxide semiconductor material of the gas sensing portion, the gas to be detected by the sensor element can be appropriately selected. The sensor element may further include a heater configured to heat the gas sensing portion.

When the sensor element is exposed to the sample gas, the detected gas contained in the sample gas and oxygen adsorbed on the surface of the gas sensing portion of the sensor element are replaced, and a reduction reaction can occur. Occurrence of the reduction reaction can remove oxygen adsorbed on the surface of the gas sensing portion. With the oxygen adsorbed on the surface of the gas sensing portion removed, the resistance value RS of the sensor element decreases, and the voltage value VS applied to the sensor element can decrease. That is, supply of the sample gas to the gas sensor 143 can decrease the voltage value VS applied to the sensor element in accordance with the concentration of the detected gas contained in the sample gas. Here, the total value of the voltage value VS and the voltage value VRL is constant. Therefore, supply of the sample gas to the gas sensor 143 can increase the voltage value VRL in accordance with the concentration of the detected gas contained in the sample gas.

The resistance element is a variable resistance element. The resistance value RL of the resistance element can vary depending on a control signal from the controller 10. One end of the resistance element is connected to the opposite end of the sensor element. The opposite end of the resistance element is connected to the ground terminal.

Adjustment of the resistance value RL of the resistance element can adjust the voltage value VS applied to the sensor element. For example, when the resistance value RL is made equal to the resistance value RS of the sensor element, the fluctuation range of the voltage value VS applied to the sensor element can be close to the maximum value.

The sensor chamber 144 is a chamber configured to internally store the gas sensor 143. As illustrated in FIG. 10, one end of the flow path 32 is connected to the sensor chamber 144. In other words, the sensor chamber 144 is connected to the first pump 132 via the flow path 32. One end of the discharge path 33 and one end of the flow path 37 are connected to the sensor chamber 144.

The discharge path 33 may include a tubular member such as a resin tube or a metal or glass pipe. One end (first end) of the discharge path 33 is connected to the sensor chamber 144, and the opposite end (second end) of the discharge path 33 opens toward the outside of a housing 30 of the gas detection device 1. By the operation of the first pump 132, the discharge path 33 discharges an exhaust gas from the sensor chamber 144 to the outside of the gas detection device 1. As illustrated in FIG. 8, a part on the opening side of the discharge path 33 can be exposed to the outside of the toilet bowl 4A.

The flow path 34 is a tubular member. One end of the flow path 34 has an opening that opens toward an outside space different from the inside of the toilet bowl 4A, and the opposite end of the flow path 34 is connected to the second valve 141. As an example, the outside is a periphery of a space where the gas detection device 1 is located, such as a space in a bathroom.

A filter 35 is a filter provided on the flow path 34. The filter 35 may be a filter that can adsorb an unnecessary component contained in the outside air sucked from the opening of the flow path 34, for example, each detected gas contained in the outside air. Since the filter 35 is the filter as described above, the outside air (purge gas) passing through the flow path 34 passes through the filter 35, whereby the content of the component of each detected gas can be reduced.

The flow path 36 has one end connected to the second valve 141, and the opposite end connected to the first valve 131. The flow path 37 has one end connected to the second valve 141 and the opposite end connected to the sensor chamber 144.

When the first pump 132 operates in a state where the first valve 131 and the second valve 141 are opened and the flow path 34, the flow path 36, and the flow path 32 communicate with one another, air (purge gas) in the bathroom is sucked from a first end of the flow path 34. The sucked purge gas is purified by passing through the filter 35, and the purified purge gas is supplied to the sensor chamber 144 through the flow path 36 and the flow path 32, and then discharged from the discharge path 33. The purge gas passes through the flow path 32 and is discharged together with the sample gas remaining in the flow path 32, whereby the flow path 32 through which the sample gas has passed is cleaned by the purge gas. When the second pump 142 operates in a state where the second valve 141 is opened and the flow path 34 and the flow path 37 communicate with each other, the purge gas in the bathroom is sucked from the opening of the flow path 34. The sucked purge gas is purified by passing through the filter 35, and the purified purge gas is supplied to the sensor chamber 144 through the flow path 37.

### Controller 10

Details of the controller 10 will be described below with reference to FIG. 9. As illustrated in FIG. 9, the controller 10 includes the main controller 101 and the detector 102. The main controller 101 controls the operation of each part of the gas detection device 1. Specifically, the main controller 101 controls operations of the subject detector 11, the defecation detector 12, the first valve 131, the first pump 132, the second valve 141, and the second pump 142. The main controller 101 operates the subject detector 11 while power is supplied to the gas detection device 1, and upon acquiring, from the subject detector 11, a signal indicating that the subject is seated on the toilet seat 4B, starts the operation of the defecation detector 12.

Upon acquiring, from the defecation detector 12, a signal indicating that feces have been excreted into the toilet bowl 4A, the main controller 101 starts collection of the sample gas in the toilet bowl 4A and detection of a predetermined component contained in the gas.

Specifically, the main controller 101 opens the first valve 131 to bring the flow path 31 and the flow path 32 into a state of communicating with each other. The main controller 101 opens the second valve 141 to bring the flow path 34 and the flow path 37 into a state of communicating with each other. In this state, the main controller 101 alternately operates the first pump 132 and the second pump 142 for a predetermined time each. Due to this, the sample gas in the toilet bowl 4A is collected from the opening at the end on the toilet bowl 4A side of the flow path 31 and is supplied to the sensor chamber 144 through the flow path 32. The purge gas is sucked from the outside and supplied to the sensor chamber 144 via the flow path 34 and the flow path 37. This enables the sensor chamber 144 to be alternately supplied with predetermined amounts of sample gas and purge gas, and the gas sensor 143 to detect a predetermined component of each detected gas contained in the respect gas and output a signal corresponding to the concentration of the predetermined component. The main controller 101 may cause the sensor chamber 144 to be supplied with the sample gas and the purge gas for 10 seconds, for example, and then cause the operations of the first pump 132 and the second pump 142 to be stopped.

Upon acquiring, from the detector 102, information indicating that the detection of the predetermined component is completed, the main controller 101 controls each part to clean the flow path 32. Specifically, the main controller 101 controls the first valve 131 and the second valve 141 to bring the flow path 34, the flow path 36, and the flow path 32 into a communicating state and operates the first pump 132. Due to this, the purge gas is supplied to the flow path 32, the sample gas remaining in the flow path 32 is discharged from the discharge path 33 through the sensor chamber 144 together with the purge gas, and cleaning of the flow path 32 is achieved. The main controller 101 controls each part to clean the sensor chamber 144. Specifically, the main controller 101 controls the second valve 141 to bring the flow path 34 and the flow path 37 into a communicating state, and operates the second pump 142. Due to this, the purge gas is supplied to the sensor chamber 144 and discharged from the discharge path 33, and cleaning of the sensor chamber 144 is achieved.

The detector 102 detects the type and concentration of a predetermined component contained in the sample gas. Specifically, the detector 102 first acquires a signal corresponding to the concentration of a predetermined component of each detected gas contained in the sample gas from the gas sensor 143. Here, the sensor chamber 144 is alternately supplied with the sample gas containing a large amount of the predetermined component and the purge gas containing a small amount of the detected gas, and therefore the intensity of the signal acquired by the detector 102 is as waveform data indicating the concentration of the predetermined component. The detector 102 estimates the type and concentration of the predetermined component based on the waveform data. For the estimation, a learned estimation model subjected to learning by a data set including a plurality of sets of waveform data as input data for learning and information indicating the type and concentration of the detected gas as training data may be used. The learning processing of this estimation model may be configured to be performed by the intestinal information estimation device 2 or may be configured to be performed by an external computer different from the intestinal information estimation device 2. The detector 102 outputs, to the communicator 16, information indicating the type and concentration of the detected predetermined component, and outputs, to the main controller 101, information indicating that the detection of the predetermined component is completed.

The detector 102 may cause the storage 15 to store the detection data D1 including each piece of detected information. The detection data D1 may include information indicating the concentration of the predetermined component. The detector 102 may cause the storage 15 to store the detection data D1 and various kinds of information relevant to the detection data D1 in association with each other. Specifically, as illustrated in FIG. 2, the detector 102 may cause the detection data D1, the subject ID and the sample gas ID indicating the subject whose sample gas has been collected, the date and time when these sample gases have been collected, and the gas detection device ID indicating the gas detection device 1 to be stored in association with one another.

### Intestinal Information Estimation Device 2

As illustrated in FIG. 9, the intestinal information estimation device 2 includes a communicator 21, a controller 22, and a storage 23, which are communication modules for communicating with the gas detection device 1 and the electronic device 3. The controller 22 controls the operation of each part of the intestinal information estimation device 2. The controller 22 includes an estimator 221 and a health information generator 222.

The storage 23 includes, for example, a semiconductor memory or a magnetic memory. The storage 23 stores various kinds of information, a program for operating the gas detection device 1, and the like. The storage 23 may function as a work memory. The storage 23 stores a learned prediction model M1 used in estimation performed by the estimator 221.

A learner 24 performs machine learning to construct the prediction model M1.

The estimator 221 inputs, to the prediction model M1, a detection signal or the concentration of a predetermined component corresponding to the detection signal, and estimates information regarding the amount and/or the existence ratio of the short-chain fatty acid-producing bacteria and/or the metabolite contained in feces of the subject. Specifically, the estimator 221 receives the detection data, the sample gas ID, the subject ID, and the like corresponding to the concentration of the predetermined component from the gas detection device 1 via the communicator 21. Based on the information, the estimator 221 estimates information regarding the amount and/or the existence ratio of the short-chain fatty acid-producing bacteria and/or the metabolite contained in feces of the subject.

The prediction model M1 may be generated in the learner 24 by machine learning processing using a combination of the following (1) and (2) as learning data.
(1) A detection signal output from the detector 102 when gases each released from a plurality of feces are supplied to the detector 102 or a concentration of a predetermined component corresponding to the detection signal
(2) Measurement information obtained by analyzing in advance and including information regarding an amount and/or an existence ratio of short-chain fatty acid-producing bacteria and/or a metabolite contained in each of the plurality of feces described in (1) FIG. 9 illustrates an aspect in which the learner 24 has a function of performing machine learning processing as an example, but the aspect is not limited to this, and the learned prediction model M1 may be introduced in advance into the intestinal information estimation device 2.

The information regarding the amount and the existence ratio of short-chain fatty acid-producing bacteria and the metabolite actually contained in each feces prepared for learning may be obtained using a next-generation sequencer for the short-chain fatty acid-producing bacteria, for example, and may be obtained using CE-MS for the metabolite. Regarding the measurement of the metabolite, another analysis technique such as GC-MS, LC-MS, or NMR may be used.

The intestinal information estimation device 2 uses the prediction model M1 generated by the machine learning as described above. According to this, from the detection signal depending on the concentration of the predetermined component, the intestinal information estimation device 2 can estimate information regarding the amount and/or the existence ratio of the short-chain fatty acid-producing bacteria and/or the metabolite contained in feces of the subject.

The estimator 221 may specifically estimate the following (A) to (H).
(A) Information regarding the amount and/or the existence ratio of Faecalibacteria is estimated from the concentration of methyl mercaptan.
(B) Information regarding the amount and/or the existence ratio of butyric acid is estimated from the concentration of hydrogen sulfide.
(C) Information regarding the amount and/or the existence ratio of bifidobacteria is estimated from the concentration of carbon dioxide and/or hydrogen.
(D) Information regarding the amount and/or the existence ratio of acetic acid is estimated from the concentration of hydrogen.
(E) Information regarding the amount and/or the existence ratio of ornithine is estimated from the concentration of carbon dioxide and/or methyl mercaptan.
(F) Information regarding the amount and/or the existence ratio of Coprococcus bacteria is estimated from the concentration of carbon dioxide.
(G) Information regarding the amount and/or the existence ratio of at least one selected from the group consisting of Streptococcus bacteria, Ruminococcus bacteria, Lachnospira bacteria, and trimethylamine is estimated from the concentration of methyl mercaptan.
(H) Information regarding the amount and/or the existence ratio of Bilophila bacteria is estimated from the concentration of 2-propanol.

The intestinal information estimation device 2 may estimate, in accordance with a preset property of the subject, information regarding the amount and/or the existence ratio of short-chain fatty acid-producing bacteria and/or a metabolite contained in feces of the subject from the detection signal depending on the concentration of the predetermined component.

The properties of the subject include the followings, for example.
Gender
Age
Presence or absence of exercise habit
Attribute (e.g., whether to be an athlete)
Presence or absence of a chronic disease (e.g., cancer or the like), diathesis (e.g., whether to be obese, whether to easily have diarrhea, or whether to easily have constipation), and presence or absence of antibiotic intake
Diet (e.g., frequency of intake of dairy products, frequency of meat-based meals, amount and frequency of intake of vegetables, and the like)
Health examination result (e.g., measurement results of height, body weight, blood pressure, and the like, stress check results, and the like)
   The health information generator 222 generates health information based on an estimation result (intestinal information) estimated by the estimator 221. The health information may be, for example, information indicating the state of the intestinal environment of the subject, specifically, an index indicating whether the intestinal environment is in a good state or a bad state. The amounts and existence ratios of the short-chain fatty acid-producing bacteria and the metabolite contained in feces reflect the amounts and existence ratios of the short-chain fatty acid-producing bacteria and the metabolite in the intestinal bacterial flora of the subject who excreted the feces. Therefore, the health information generator 222 may generate an index indicating the composition of bacteria in the intestinal bacterial flora estimated from the amounts and existence ratios of the short-chain fatty acid-producing bacteria and the metabolite contained in feces of the subject, for example, the balance between good bacteria and bad bacteria. The health information generator 222 may generate an index indicating the physical condition, the health condition, the immunity, the tendency to get fat, and the like of the subject that can be estimated from the intestinal environment of the subject based on the information described above. Furthermore, in order to improve the intestinal environment of the subject, the health information generator 222 may output information indicating advice prompting diet, exercise, and the like. The health information may include evaluation, useful information, and remarks. The health information generator 222 transmits each piece of the generated information to the electronic device 3 via the communicator 21. The health information generator 222 may cause the storage 23 to store, in association with the subject ID and the sample gas ID, the estimation result information including the intestinal information estimated by the estimator 221.

The storage 23 may store a plurality of prediction models M1 for each property (attribute) of the subject. For example, the storage 23 may store the plurality of prediction models M1 corresponding to at least one selected from the group consisting of gender, age, presence or absence of exercise habit, and dietary habit as the property (attribute) of the subject. The estimator 221 may estimate, in accordance with the property of the subject, information regarding the amount and/or the existence ratio of short-chain fatty acid-producing bacteria and/or a metabolite contained in feces of the subject using any one of the plurality of prediction models M1 stored in the storage 23. For example, the estimator 221 may select any one of the plurality of prediction models M1 in accordance with the gender of the subject.

The prediction model M1 may be generated by the learner 24 using, as learning data, the property (attribute) of a person who has excreted each of feces prepared for learning, and information regarding the amounts and the existence ratios of the short-chain fatty acid-producing bacteria and/or the metabolite contained in the feces. The estimator 221 may estimate information regarding the amount and/or the existence ratio of short-chain fatty acid-producing bacteria and/or a metabolite contained in feces of the subject by inputting, to the prediction model M1, information regarding the property of the subject in addition to the detection signal or the concentration of the predetermined component corresponding to the detection signal.

The subject information held by the intestinal information estimation device 2 may include information regarding the property (attribute) of the subject. The estimator 221 may perform estimation using any one of the plurality of prediction models M1 in accordance with the property of the subject included in the subject information corresponding to the individual specified and identified by the subject detector 11.

### Electronic Device 3

As illustrated in FIG. 9, the electronic device 3 includes a communicator 311, which is a communication module for communicating with the intestinal information estimation device 2, a controller 312 configured to control the operation of each part of the electronic device 3, and a display 313. The controller 312 can receive, via the communicator 311 by wireless communication or wired communication, the estimation result or the health information output by the intestinal information estimation device 2. The electronic device 3 can display the received estimation result or health information on the display 313. The display 313 may include a display that can display characters and the like and a touchscreen that can detect contact of a finger or the like of the user (subject). The display may include a display device such as a liquid crystal display (LCD), an organic electro-luminescence display (OELD), or an inorganic electro-luminescence display (IELD). A detection method of the touchscreen may be any method such as a capacitive method, a resistance film method, a surface elastic wave method (or an ultrasonic method), an infrared method, an electromagnetic inductive method, or a load detection method.

### Example of Flow of Processing of Intestinal Information Estimation System 100

The flow of processing (gas detection method) performed in the intestinal information estimation system 100 will be described with reference to FIG. 11. FIG. 11 is a flowchart showing an example of the flow of processing performed in the intestinal information estimation system 100. In the following description, assume that the gas detection device 1 is configured to include a pressure sensor as each of the subject detector 11 and the defecation detector 12.

First, when the subject sits on the toilet seat 4B to excrete feces to the toilet 4, the subject detector 11 outputs, to the main controller 101, a signal indicating that the subject is seated on the toilet seat 4B. Upon acquiring the signal, the main controller 101 detects that the subject sits on the toilet seat 4B (S1), starts the operation of the defecation detector 12, and waits until the defecation of the subject is detected (S2). The defecation detector 12 outputs, to the main controller 101, a signal indicating that excretion of the specimen from the subject (defecation of the subject) has been detected. Upon acquiring the signal (YES in S2), the main controller 101 controls the first valve 131 to bring the flow path 31 and the flow path 32 into a state of communicating with each other.

The main controller 101 operates the first pump 132 to cause the sample gas to be collected from the opening on the toilet bowl 4A side of the flow path 31 (S3) and the sample gas to be supplied to the sensor chamber 144 (S4). The main controller 101 operates the first pump 132 for a predetermined time to cause a predetermined amount of a first sample gas to be supplied to the sensor chamber 144, and then stops the first pump 132. The main controller 101 controls the first valve 131 to bring the flow path 31 and the flow path 32 into a state of not communicating with each other. Thereafter, the main controller 101 controls the second valve 141 and the second pump 142 to cause the purge gas in the bathroom to be sucked from the flow path 34 and to be supplied to the sensor chamber 144. The main controller 101 performs, for about 10 seconds in total, alternately supply of the first sample gas to the sensor chamber 144 by the first pump 132 and supply of the purge gas to the sensor chamber 144 by the second pump 142.

The detector 102 detects each of the predetermined components (at least one selected from the group consisting of methyl mercaptan, hydrogen sulfide, and carbon dioxide) contained in the sample gas, and outputs a detection signal depending on the predetermined component (S5: detection step). The detector 102 transmits a detection signal depending on the concentration of the predetermined component contained in the detected sample gas to the intestinal information estimation device 2 via the communicator 16. The detector 102 outputs, to the main controller 101, information indicating that the first detection step is completed.

Upon acquiring the information indicating that the detection step is completed, the main controller 101 may control the first valve 131, the first pump 132, the second valve 141, and the second pump 142 to perform cleaning of the flow path 32 and the sensor chamber 144.

The estimator 221 of the intestinal information estimation device 2 receives a detection signal depending on the concentration of a predetermined component from the gas detection device 1 via the communicator 21. From the detection signal depending on the concentration of the predetermined component or the concentration of the predetermined component corresponding to the detection signal, the estimator 221 estimates information regarding the amount and/or the existence ratio of the short-chain fatty acid-producing bacteria and/or the metabolite contained in feces of the subject (S6: estimation step). The estimator 221 outputs the estimated intestinal information.

The health information generator 222 generates health information regarding the health condition of the subject based on the intestinal information estimated by the estimator 221 (S7). The health information generator 222 transmits the estimation result information including the intestinal information and the health information to the electronic device 3 via the communicator 21.

The controller 312 of the electronic device 3 receives, from the intestinal information estimation device 2 via the communicator 311, the estimation result information including the intestinal information estimated based on the predetermined component contained in the gas released from feces and the health information generated based on the intestinal information. The controller 312 notifies the subject of the received estimation result information by displaying it on the display 313, for example.

### Effects of Intestinal Information Estimation System 100

As described above, an intestinal information estimation method according to the present embodiment includes the detection step (S5) of outputting a detection signal depending on the concentration of a predetermined component (at least one selected from the group consisting of methyl mercaptan, hydrogen sulfide, and carbon dioxide) from the gas released from feces excreted from the subject. The intestinal information estimation method according to the present embodiment includes the estimation step (S6) of estimating information regarding the amount and/or the existence ratio of the short-chain fatty acid-producing bacteria and/or the metabolite contained in feces of the subject.

Based on the concentration of a predetermined component detected from the gas released from feces of the subject, the intestinal information estimation system 100 estimates information regarding the amount and/or the existence ratio of the short-chain fatty acid-producing bacteria and/or the metabolite contained in feces of the subject. The predetermined component is at least one selected from the group consisting of methyl mercaptan, hydrogen sulfide, and carbon dioxide. This enables the intestinal information estimation system 100 to easily and accurately estimate the information regarding the inside of the intestine of the subject.

### Variation

In the intestinal information estimation system 100 in the above-described embodiment, the gas detection device 1 detects a predetermined component contained in a gas, and outputs a detection signal depending on the concentration of the predetermined component. The intestinal information estimation device 2 estimates information regarding an amount and/or an existence ratio of short-chain fatty acid-producing bacteria and/or a metabolite contained in feces of the subject. However, the intestinal information estimation system 100 is not limited to this configuration. For example, the gas detection device 1 may include the estimator 221 and may perform the processing performed in the intestinal information estimation device 2. In this case, the estimation of the information regarding the amounts and existence ratios of the short-chain fatty acid-producing bacteria and/or the metabolite contained in feces of the subject from collection of the sample gas can be completed only by the gas detection device 1. In this case, the intestinal information estimation system 100 needs not include the intestinal information estimation device 2, and the gas detection device 1 may transmit the estimated information to the electronic device 3.

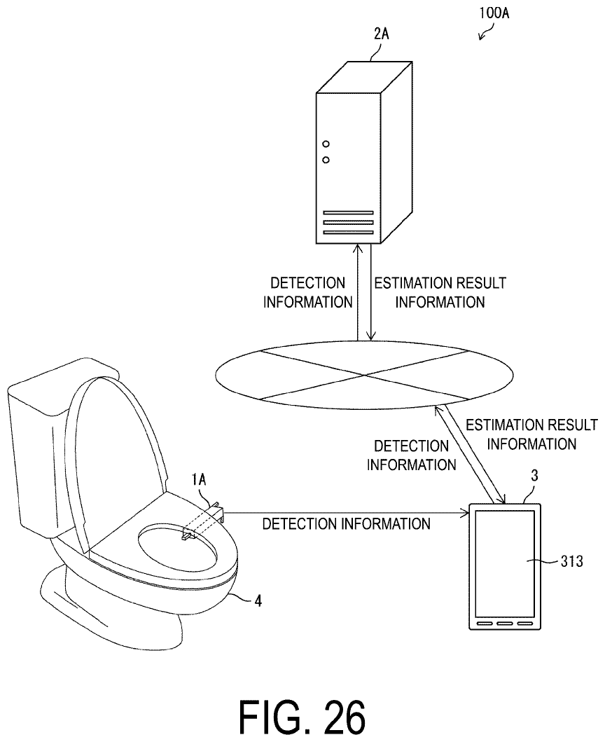

FIG. 26 is a schematic diagram illustrating the configuration of an intestinal information estimation system 100A, which is a variation of the intestinal information estimation system 100. As illustrated in FIG. 26, the intestinal information estimation system 100A includes a gas detection device 1A and an intestinal information estimation device 2A in place of the gas detection device 1 and the intestinal information estimation device 2. As illustrated in FIG. 26, the gas detection device 1A needs not be communicably connected to the intestinal information estimation device 2A via a communication network. In the intestinal information estimation system 100A, the gas detection device 1A is communicably connected to only the electronic device 3. In this case, the gas detection device 1A may transmit various kinds of information such as concentration information to the electronic device 3, and the electronic device 3 may transmit, to the intestinal information estimation device 2A, the concentration information and the like received from the gas detection device 1A. As an example, the gas detection device 1A transmits concentration information to the electronic device 3 via a communication device such as a LAN. The electronic device 3 transmits detection information to the intestinal information estimation device 2A. The intestinal information estimation device 2A transmits estimation result information to the electronic device 3 that is the transmission source of the detection information.

### Implementation Example by Software

The functions of the intestinal information estimation systems 100 and 100A (hereinafter, called a “system”) can be implemented by a program for causing a computer to function as the system, the program for causing a computer to function as each control block (in particular, each part included in the controllers 10, 10A, and 22) of the system.

In this case, the system includes a computer having at least one control device (e.g., processor) and at least one storage device (e.g., memory) as hardware for executing the program. By executing the program by the control device and the storage device, the functions described in the embodiment are implemented.

The program may be recorded on one or more computer-readable non-transitory recording media. This recording media may be or need not be included in the device. In the latter case, the program may be supplied to the device via any wired or wireless transmission medium.

Some or all of the functions of the control blocks can also be implemented by logic circuits. For example, an integrated circuit in which logic circuits functioning as the control blocks are formed is also included in the scope of the present disclosure. In addition to this, for example, a quantum computer can implement the functions of the control blocks.

The invention according to the present disclosure has been described above based on the drawings and examples. However, the invention according to the present disclosure is not limited to each embodiment described above. That is, the invention according to the present disclosure can be varied in various ways within the scope presented in the present disclosure, and embodiments obtained by appropriately combining the technical means disclosed in different embodiments are also included in the technical scope of the invention according to the present disclosure. In other words, note that a person skilled in the art can easily make various variations or modifications based on the present disclosure. Note that these variations or modifications are included within the scope of the present disclosure.

### Example 1

An example of the present disclosure will be described below.

### Estimation by Intestinal Information Estimation System 100

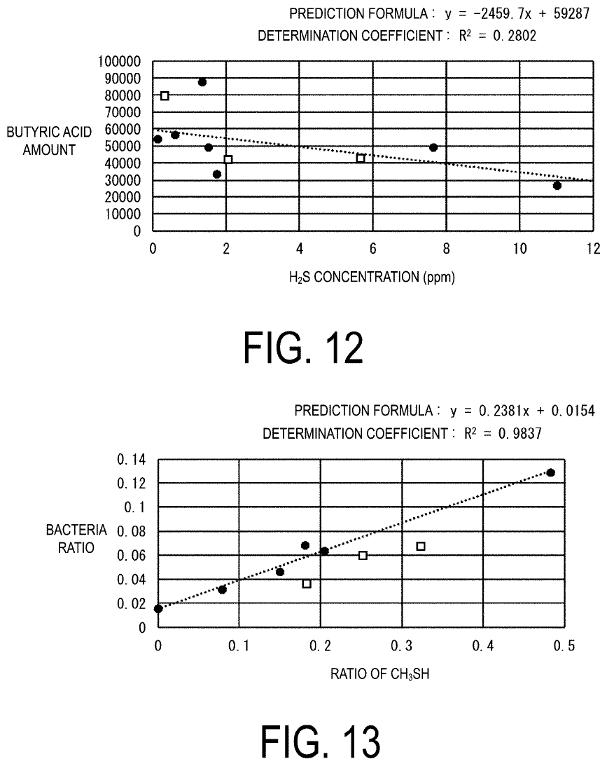

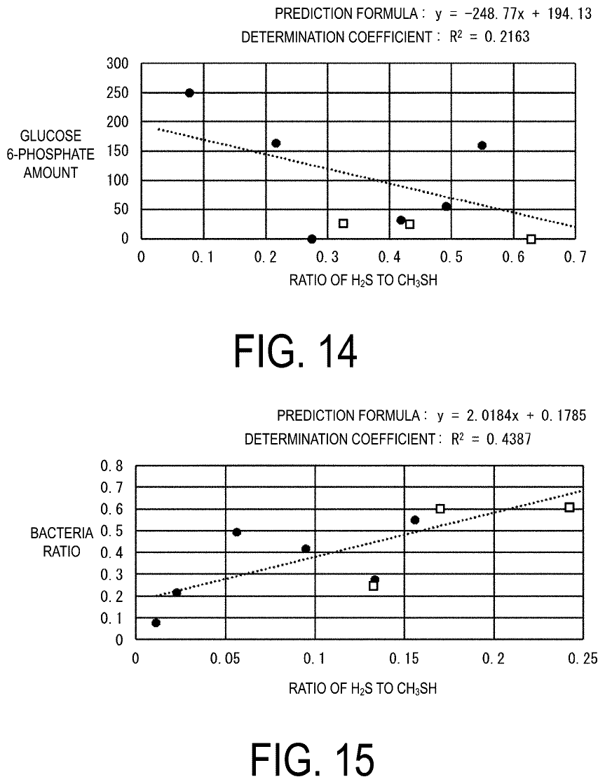

(1) 60 g of feces of seven subjects were collected as feces for learning. The gas released from each feces was supplied to the gas detection device 1, and the butyric acid amount (unit: nmol/g) contained in the feces of the subject was estimated by the intestinal information estimation device 2 from the concentration (unit: ppm) of H₂S contained in the sample gas output from the detector 102. In FIG. 12, the butyric acid amount with respect to the concentration of H₂S of each sample gas was plotted with “●”. Using the plotted result, a regression line was obtained from the least squares method, and a prediction formula was obtained from the regression line (dotted line).
(2) 60 g of feces of six subjects were collected as feces for learning. The gas released from each feces was supplied to the gas detection device 1, and the ratio of the sum of Ruminococcus bacteria and Lachnospira bacteria contained in the feces to the mass of the feces of the subject was estimated by the intestinal information estimation device 2 from the concentration ratio of CH₃SH to the entire gas contained in the sample gas output from the detector 102. In FIG. 13, the Ruminococcus bacteria ratio to the concentration ratio of CH₃SH of each sample gas was plotted with “●”. Using the plotted result, a regression line was obtained from the least squares method, and a prediction formula was obtained from the regression line (dotted line).
(3) 60 g of feces of six subjects were collected as feces for learning. The gas released from each feces was supplied to the gas detection device 1. The glucose 6-phosphate amount (unit: nmol/g) contained in the feces of the subject was estimated by the intestinal information estimation device 2 from the ratio of the sum of H₂S and CH₃SH to the entire gas contained in the sample gas output from the detector 102. In FIG. 14, the glucose 6-phosphate amount with respect to the ratio of the sum of H₂S and CH₃SH to the entire gas contained in each sample gas was plotted with “●”. Using the plotted result, a regression line was obtained from the least squares method, and a prediction formula was obtained from the regression line (dotted line).
(4) 60 g of feces of six subjects were collected as feces for learning. The gas released from each feces was supplied to the gas detection device 1. The ratio of the sum of Faecalibacteria and Lachnospira bacteria contained in the feces of the subject was estimated by the intestinal information estimation device 2 from the ratio of the sum of H₂S and CH₃SH to the entire gas contained in the sample gas output from the detector 102. In FIG. 15, the ratio of the sum of Faecalibacteria and Lachnospira bacteria to the ratio of the sum of H₂S and CH₃SH to the entire gas contained in each sample gas was plotted with “●”. Using the plotted result, a regression line was obtained from the least squares method, and a prediction formula was obtained from the regression line (dotted line).

### Verification

In order to verify the obtained prediction formulae (1) to (4), 60 g of feces of each of subjects A, B, and C was collected, and the concentration or the concentration ratio of a predetermined component contained in the sample gas released from each feces was measured. The information regarding the amount and the existence ratio of short-chain fatty acid-producing bacteria and the metabolite actually contained in each feces was obtained using a next generation sequencer for the existence ratio of the short-chain fatty acid-producing bacteria, and the amount and the existence ratio of the metabolite were obtained using CE-MS. The information regarding the amount and the existence ratio of the metabolite may be obtained by using another analysis technique such as GC-MS, LC-MS, or NMR regarding the measurement of the metabolite.

The actually obtained data (correct value) corresponds to “□” plotted in FIGS. 12 to 15. An intersection point with the regression line when a straight line parallel to the y axis is drawn from each point of the correct value toward the regression line corresponds to a predicted value. Table 1 shows the result of obtaining the difference (residual) between each correct value and the predicted value and calculating the ratio of each residual with respect to the measurement range (difference between the maximum value and the minimum value of the measurement data).

TABLE 1

| | RATIO OF RESIDUAL TO MEASUREMENT RANGE (%) | | | |
|---|---|---|---|---|
| SUBJECT | (1) | (2) | (3) | (4) |
| A | 35.1340 | 21.9442 | 24.2028 | 44.6070 |
| B | 5.0678 | 19.7987 | 15.0680 | 20.2879 |
| C | 19.7813 | 13.6407 | 34.3967 | 4.5904 |

From Table 1, the ratio of each residual was about 45% even in one with the worst accuracy. It can be said that the smaller the ratio of the residual is, the higher the prediction accuracy indicated by the regression line is. This has proved that the amounts and the existence ratios of the short-chain fatty acid-producing bacteria and the metabolite estimated by the intestinal information estimation system 100 have high accuracy.

### Other Estimation by Intestinal Information Estimation System 100

Also regarding the following (5) to (13), a predetermined component was detected from a gas released from feces of the subject using the intestinal information estimation system 100, and the amounts and the existence ratios of short-chain fatty acid-producing bacteria and a metabolite were estimated from the concentration of a predetermined component. Regarding (11), as the property of the subject, gender is limited, and estimation was performed using data of only female. As described above, it is clear that the amounts and the existence ratios of short-chain fatty acid-producing bacteria and a metabolite can be estimated using the intestinal information estimation system 100 from the regression line obtained from each plot.

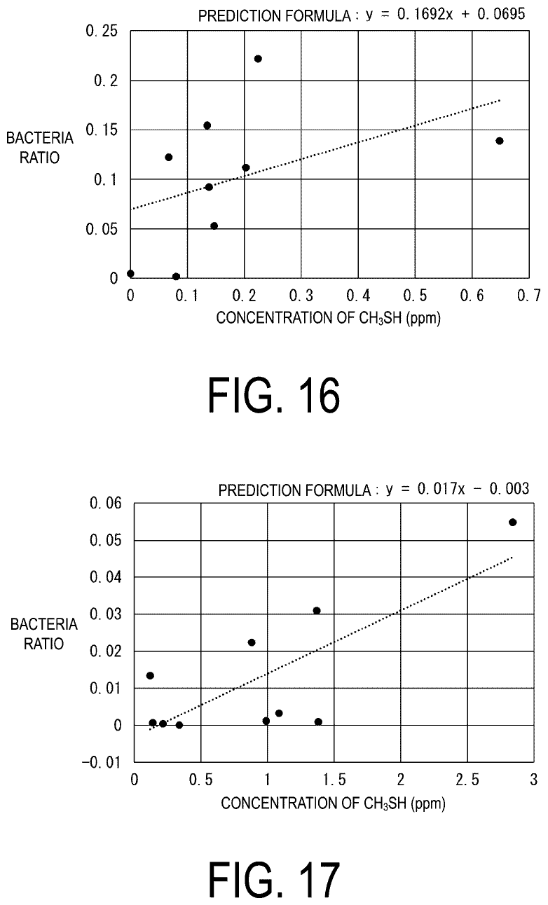

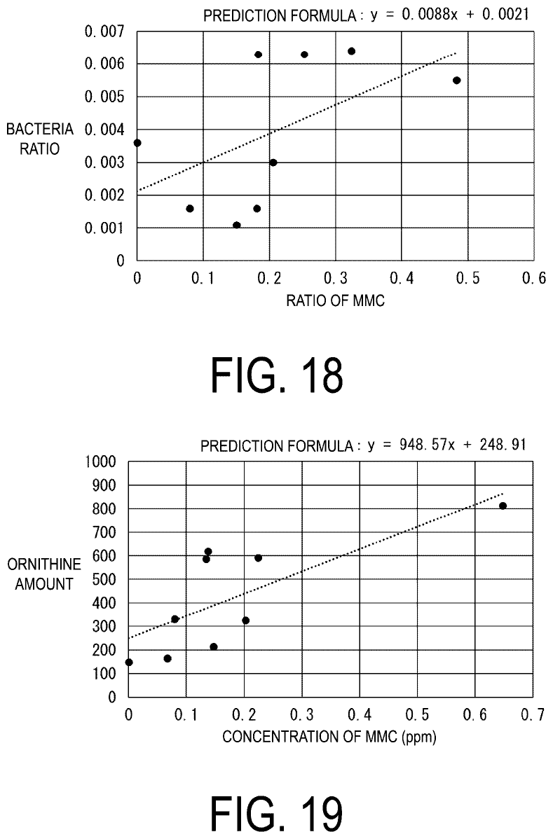

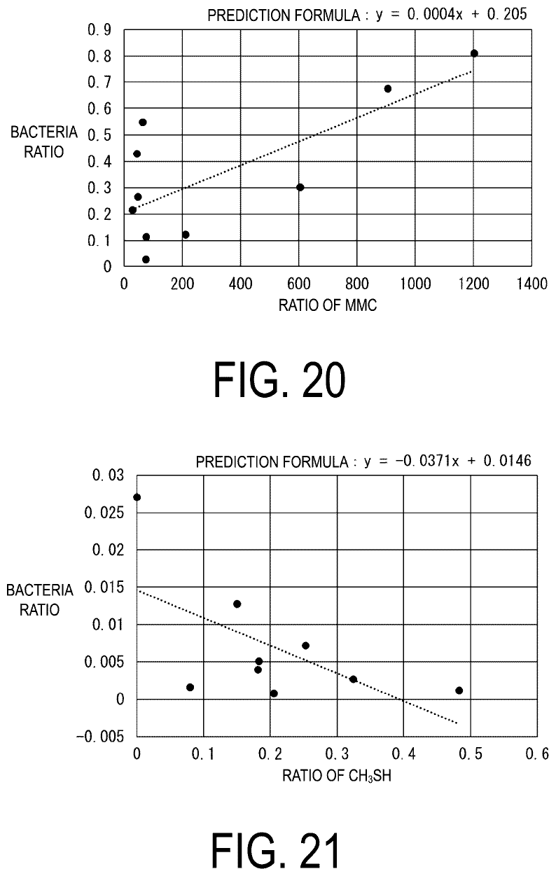

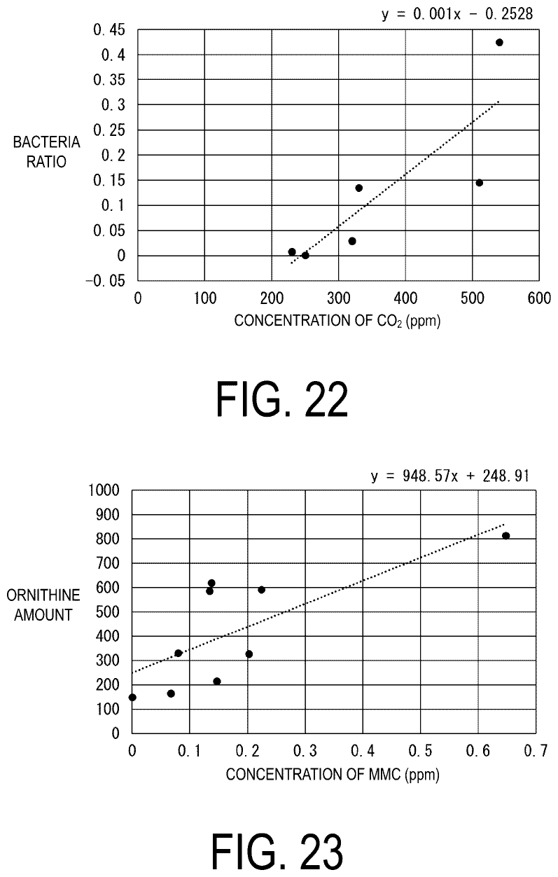

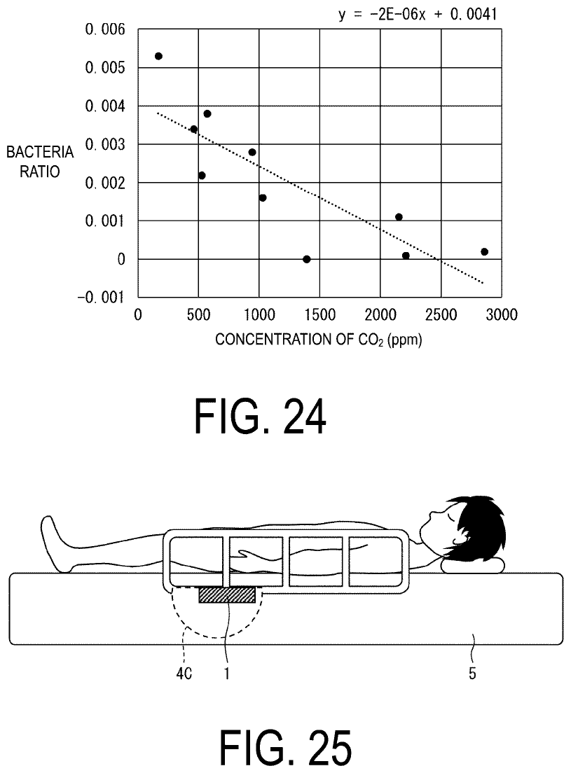

(5) Faecalibacteria ratio was estimated from CH₃SH concentration (ppm) (FIG. 16)
(6) Ruminococcus bacteria ratio was estimated from CH₃SH concentration (ppm) (FIG. 17)
(7) Lachnospira bacteria ratio was estimated from ratio of CH₃SH (FIG. 18)
(8) Ornithine amount (unit: nmol/g) was estimated from CH₃SH concentration (ppm) (FIG. 19)
(9) Trimethylamine amount (unit: nmol/g) was estimated from ratio of CH₃SH (FIG. 20)
(10) Streptococcus bacteria ratio was estimated from ratio of CH₃SH (FIG. 21)
(11) Bifidobacteria ratio was estimated from CO₂ concentration (ppm) (FIG. 22)
(12) Ornithine amount (unit: nmol/g) was estimated from CH₃SH concentration (ppm) (FIG. 23)
(13) Coprococcus bacteria ratio was estimated from CO₂ concentration (ppm) (FIG. 24)

### REFERENCE SIGNS

1, 1A Gas detection device
2, 2A Intestinal information estimation device
3 Electronic device
4 Toilet
102 Detector
221 Estimator
222 Health information generator

1. An intestinal information estimation system, comprising:
a detector configured to detect a predetermined component from a gas released from feces of a subject and output a detection signal depending on a concentration of the predetermined component; and
an estimator configured to input the detection signal or the concentration of the predetermined component corresponding to the detection signal to a prediction model and estimate information regarding an amount and/or an existence ratio of short-chain fatty acid-producing bacteria and/or a metabolite contained in the feces of the subject,
wherein the predetermined component is at least one selected from the group consisting of methyl mercaptan, hydrogen sulfide, hydrogen, and carbon dioxide.

2. The intestinal information estimation system according to claim 1, wherein the prediction model is generated by machine learning using learning data including a combination of (1) a detection signal output from the detector when gases each released from a plurality of feces are supplied to the detector or a concentration of the predetermined component corresponding to the detection signal and (2) measurement information obtained by analyzing in advance and including information regarding an amount and/or an existence ratio of short-chain fatty acid-producing bacteria and/or a metabolite contained in each of the plurality of feces.

3. The intestinal information estimation system according to claim 1, wherein the short-chain fatty acid-producing bacteria are butyric acid-producing bacteria and/or acetic acid-producing bacteria.

4. The intestinal information estimation system according to claim 1, wherein the metabolite is butyric acid and/or acetic acid.

5. The intestinal information estimation system according to claim 1, wherein information regarding an amount and/or an existence ratio of Faecalibacteria is estimated from a concentration of methyl mercaptan detected from the gas released from the feces of the subject.

6. The intestinal information estimation system according to claim 1, wherein information regarding an amount and/or an existence ratio of butyric acid is estimated from a concentration of hydrogen sulfide detected from the gas released from the feces of the subject.

7. The intestinal information estimation system according to claim 1, wherein information regarding an amount and/or an existence ratio of bifidobacteria is estimated from a concentration of carbon dioxide and/or hydrogen detected from the gas released from the feces of the subject.

8. The intestinal information estimation system according to claim 1, wherein information regarding an amount and/or an existence ratio of acetic acid is estimated from a concentration of hydrogen detected from the gas released from the feces of the subject.

9. The intestinal information estimation system according claim 1, wherein information regarding an amount and/or an existence ratio of ornithine is estimated from a concentration of carbon dioxide and/or the methyl mercaptan detected from the gas released from the feces of the subject.

10. The intestinal information estimation system according to claim 1, wherein information regarding an amount and/or an existence ratio of Coprococcus bacteria is estimated from a concentration of carbon dioxide detected from the gas released from the feces of the subject.

11. The intestinal information estimation system according to claim 1, wherein information regarding an amount and/or an existence ratio of at least one selected from the group consisting of Streptococcus bacteria, Ruminococcus bacteria, Lachnospira bacteria, and trimethylamine is estimated from a concentration of methyl mercaptan detected from the gas released from the feces of the subject.

12. The intestinal information estimation system according to claim 1,
wherein the detector can detect 2-propanol from the gas released from the feces of the subject, and
information regarding an amount and/or an existence ratio of Bilophila bacteria is estimated from a concentration of 2-propanol detected from the gas released from the feces of the subject.

13. The intestinal information estimation system according to claim 1 further comprising
a health information generator configured to generate health information based on an estimation result from the estimator.

14. The intestinal information estimation system according to claim 1, wherein the detector is installed in a toilet of a bathroom.

15. The intestinal information estimation system according to claim 1, wherein the detector is installed in a bed for a person requiring nursing care.

16. The intestinal information estimation system according to claim 1, wherein the detector can be carried by the subject.

17. The intestinal information estimation system according to claim 1, wherein the estimator inputs the detection signal or a concentration of the predetermined component corresponding to the detection signal to a prediction model depending on a property of the subject.

18. The intestinal information estimation system according to claim 1, wherein the estimator inputs information regarding a property of the subject to a prediction model.
